(19) United States
(12) Patent Application Publication
(10) Pub. No.: US 2013/0084627 A1
(43) Pub. Date: Apr. 4, 2013

(54) BIOCHIP

(71) Applicants: Jeong Suong YANG, Yongin (KR); Bo Sung Ku, Suwon (KR)

(72) Inventors: Jeong Suong YANG, Yongin (KR); Bo Sung Ku, Suwon (KR)

(73) Assignee: SAMSUNG ELECTRO-MECHANICS CO.,LTD.

(21) Appl. No.: 13/361,650

(22) Filed: Jan. 30, 2012

(30) Foreign Application Priority Data
Sep. 30, 2011 (KR) 10-2011-0099782

(51) Int. Cl. C12M 1/00

(52) U.S. Cl. 435/283.1

(57) ABSTRACT

There is provided a biochip including a first substrate having micro wells formed to a predetermined depth and partitioned by a plurality of barrier ribs, and a reservoir formed below the micro wells and receiving liquid; and a second substrate coupled with the first substrate to thereby allow biomaterials provided thereon to be inserted into the micro wells while having a predetermined interval therebetween.

### CROSS-REFERENCE TO RELATED APPLICATIONS

This application claims the priority of Korean Patent Application No. 10-2011-0099782 filed on Sep. 30, 2011, in the Korean Intellectual Property Office, the disclosure of which is incorporated herein by reference.

### BACKGROUND OF THE INVENTION

1. Field of the Invention

The present invention relates to a biochip, and more particularly, to a biochip having excellent measurement efficiency and accuracy.

2. Description of the Related Art

Recently, demands for biomedical equipment and biotechnological methods for rapidly diagnosing various human diseases have increased. Therefore, development of a biosensor or a biochip capable of providing test results within a short time frame that can replace existing tests for specific diseases performed over a relatively long period in a hospital or a laboratory has been actively undertaken.

Biosensor or biochip technology is needed in hospitals, pharmaceutical companies, cosmetics companies, and the like. A method of testing the validity and safety (toxicity) of specific drugs by testing cell reaction to specific drugs has been used in the pharmaceutical and cosmetics fields. The existing method uses animal test subjects or a large amount of reagent, and thus, may be time consuming and costly.

Therefore, a need exists for the development of a biosensor or biochip capable of performing rapid and accurate diagnoses while saving on costs.

Biochips may be divided into a DNA chip, a protein chip, and a cell chip, according to the types of biomaterials intended to be fixed thereto. In the early stages of biochip development, DNA chips attracted great interest in line with advances in knowledge of human genetic information. However, protein chips and cell chips have stirred new interest due to an increased focus on proteins and protein binders that are sources of vital body activities, that is, cells that are the nucleus of life.

Protein chips encountered a problem known as “non-selective adsorption” at an early stage of development, but several noticeable research results associated therewith have recently appeared.

Cell chips have been prominent as an effective medium applicable to various fields such as in the development of new medicines, genomics, proteomics, or the like.

### SUMMARY OF THE INVENTION

An object of the present invention is to provide a biochip having excellent measurement efficiency and accuracy.

According to an embodiment of the present invention, there is provided a biochip, including: a first substrate having micro wells formed to a predetermined depth and partitioned by a plurality of barrier ribs, and a reservoir formed below the micro wells and receiving liquid; and a second substrate coupled with the first substrate to thereby allow biomaterials provided thereon to be inserted into the micro wells while having a predetermined interval therebetween.

The barrier ribs may be provided with through holes communicating with the reservoir.

The reservoir may have a single receiving part provided to correspond to the plurality of micro wells.

The reservoir may have a plurality of receiving parts corresponding to the plurality of micro wells, respectively.

The reservoir may have a plurality of receiving parts, each of which may be provided to correspond to the plurality of micro wells.

The liquid may be water or deionized water.

The biomaterials may be dispersed in a porous dispersion material and be disposed on the second substrate.

The second substrate may be provided with a plurality of micro fillers having a predetermined interval therebetween, and the biomaterials may be disposed on one surface of each of the micro fillers.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a perspective view schematically showing a biochip according to an embodiment of the present invention;

FIG. 2 is a cross-sectional view schematically showing a first substrate according to an embodiment of the present invention;

FIG. 3 is a cross-sectional view schematically showing a second substrate according to an embodiment of the present invention;

FIG. 4 is a cross-sectional view schematically showing a state in which a first substrate and a second substrate are coupled to configure a biochip according to an embodiment of the present invention; and

FIG. 5 is a cross-sectional view schematically showing a biochip according to another embodiment of the present invention.

### DETAILED DESCRIPTION OF THE INVENTION

Embodiments of the present invention will now be described in detail with reference to the accompanying drawings. The embodiments of the present invention maybe modified in many different forms and the scope of the invention should not be limited to the embodiments set forth herein. Rather, these embodiments are provided so that this disclosure will be thorough and complete, and will fully convey the concept of the invention to those skilled in the art. In the drawings, the shapes and dimensions of components may be exaggerated for clarity, and the same reference numerals will be used throughout to designate the same or like components.

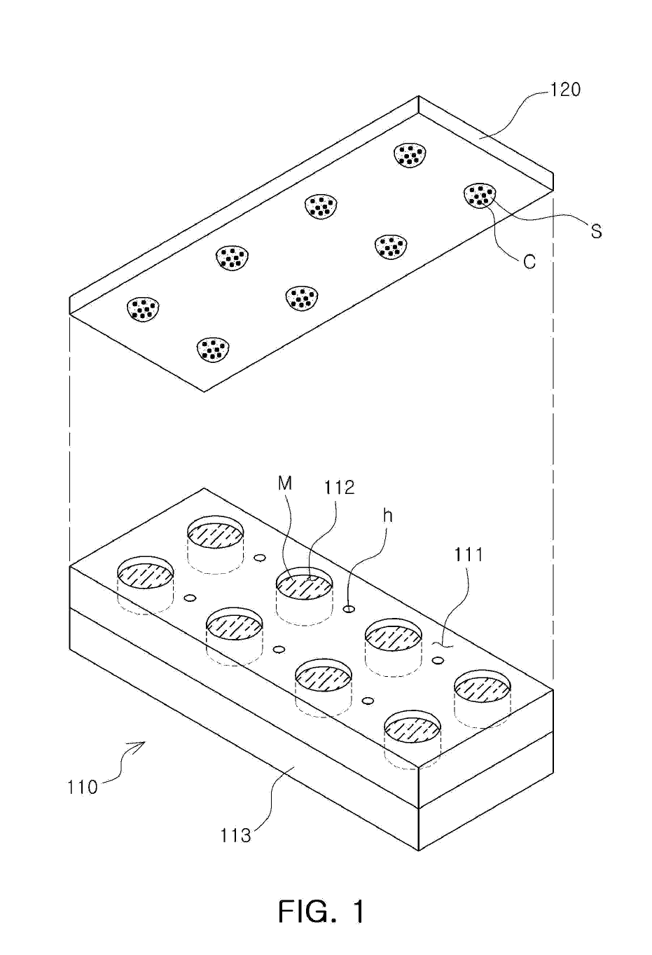

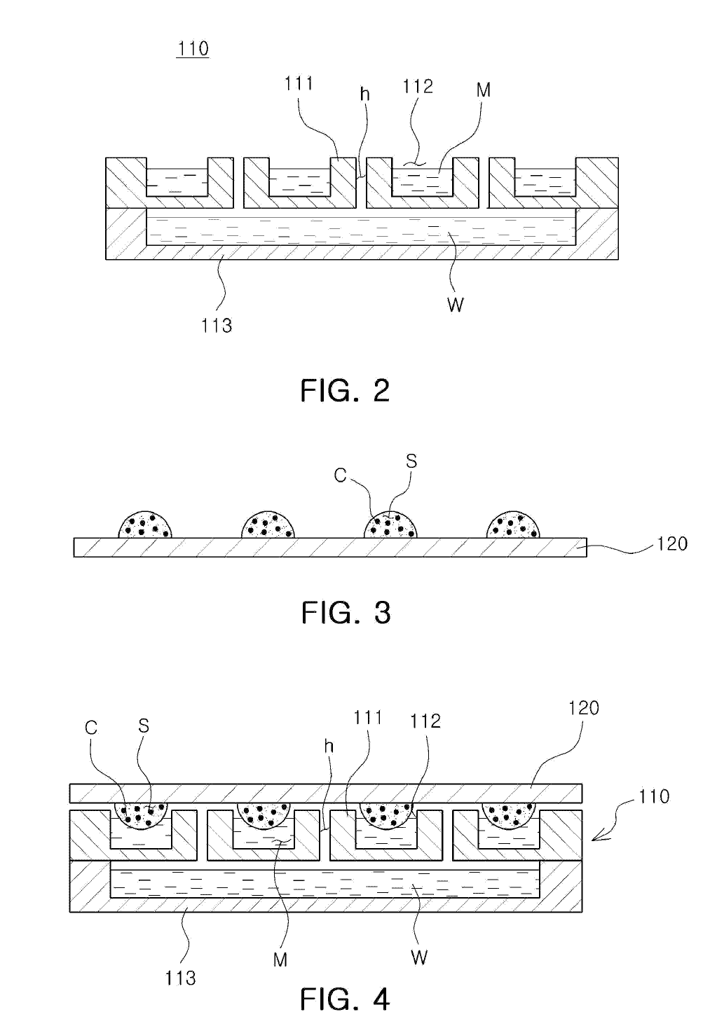

FIG. 1 is a perspective view schematically a biochip according to an embodiment of the present invention. FIG. 2 is a cross-sectional view schematically showing a first substrate according to the embodiment of the present invention and FIG. 3 is a cross-sectional view schematically showing a second substrate according to the embodiment of the present invention. FIG. 4 is a cross-sectional view schematically showing a state in which the first substrate and the second substrate are coupled to configure the biochip according to the embodiment of the present invention.

Referring to FIGS. 1 through 4, the biochip according to the embodiment of the present invention may include a first substrate 110 having a plurality of barrier ribs 111, micro wells 112, and a reservoir 113 and a second substrate 120.

As shown in FIG. 2, the first substrate 110 may be provided with the plurality of micro wells 112 having a predetermined interval from one another, and the micro wells 112 maybe partitioned by the barrier ribs 111. The micro well 112 is formed to a predetermined depth from one surface of the first substrate and may be understood as a micro groove.

A diameter of the micro well 112 may be on a micrometer level. Without being limited thereto, the diameter of the micro well 112 may be 50 to 1000 μm. According to the embodiment of the present invention, the micro wells 112 may be highly integrated in the first substrate 110 and the interval between the micro wells 112 may be 50 to 1000 μm without being limited thereto. The interval between the micro wells 112 maybe a width of the barrier rib 111.

A reagent M may be injected into the micro well 112. The reagent M is not specifically limited and, for example, may be a cell culture solution, specific drugs or various aqueous solutions.

The first substrate 110 may be made of polymer without being limited thereto. Types of polymer are not particularly limited. For example, polymethylmetacrylate (PMMA), polycarbonate (PC), polyethylene, or the like, and a mixture thereof may be used.

Various reagents M may be contained in the micro wells 112 formed in the first substrate 110.

When the biomaterials are inserted into the micro wells, various reagents may be directly supplied to the biomaterials. Therefore, the cells may be cultivated and various experiments may be performed by analyzing the characteristics of the biomaterials with regard to the reagents.

The reservoir 113 may be formed on a lower portion of the first substrate 110. The reservoir 113 includes a receiving part having a predetermined depth, and the receiving part may contain liquid. A vapor pressure of the biochip may be maintained by the liquid. Here, the liquid may evaporate at an appropriate speed at a temperature at which cell culturing and various experiments maybe preformed. The liquid may be water W or deionized water without being limited thereto.

The reservoir 113 may be formed below the micro well 112. The reservoir 113 maybe formed so as to contact the bottom surface of the micro well.

According to the embodiment of the present invention, the reservoir 113 may be formed over the entirety of the first substrate 110.

In addition, according to the embodiment of the present invention, the reservoir 113 may be provided to correspond to the plurality of micro wells. The reservoir may have a single receiving part to correspond to all of the micro wells formed in the first substrate.

Alternatively, the reservoir may have a plurality of receiving parts, each of which may be provided to correspond to a single micro well or may be provided to correspond to a plurality of micro wells.

According to the embodiment of the present invention, as shown in FIG. 2, the barrier ribs 111 may be provided with through holes h communicating with the reservoir 113. The water W received in the reservoir 113 may be evaporated through the through holes h.

Referring to FIG. 3, the second substrate 120 according to the embodiment of the present invention maybe provided with the biomaterials C. The biomaterials C may be arranged on the second substrate 120 having a predetermined interval therebetween. The biomaterials maybe arranged in a matrix form.

The biomaterials C may be arranged on the second substrate 120, while corresponding to the positions of the micro wells 112 formed in the first substrate 110, such that the biomaterials C may be inserted into the respective micro wells 112 formed in the first substrate 110.

The biomaterials C may be highly integrated on the second substrate 120 and the interval between the biomaterials may be 50 to 1000 μm without being limited thereto.

According to the embodiment of the present invention, the biomaterial C may be dispersed in a dispersion material S capable of stably maintaining a biomaterial tissue, to thereby be attached to the second substrate 120.

Types of the biomaterials C are not specifically limited and, for example, may be nucleic acid arrangement such as RNA, DNA, or the like, peptide, protein, lipid, organic or inorganic chemical molecules, virus particles, procaryotic cells, organelle, or the like. In addition, the types of cells are not particularly limited and may be, for example, microorganism, plant and animal cells, cancer cells, neuron cells, cells in blood vessel, immune cells, or the like.

The dispersion material S may be a porous material through which the reagent M such as a culture solution, specific drugs, various aqueous solutions, or the like, may be transmitted.

The dispersion material S is not limited thereto and, for example, sol-gel, hydro gel, alginate gel, organogel, xerogel, gelatin, collagen, or the like may be used.

According to the embodiment of the present invention, the biomaterial C is dispersed in the dispersion material S and thus, may be attached to the second substrate 120 in a three-dimensional structure. The biomaterial having the three-dimensional structure is more similar to a bioenvironment and thus, more accurate results may be obtained.

The second substrate 120 may be formed of polymer without being limited thereto. Types of polymer are not particularly limited. For example, polystyrene (PS), polycarbonate (PC), polyethylene, poly (styrene-maleic anhydride) (PSMA) or the like, and a mixture thereof may be used.

The polymer having the poly (styrene-maleic anhydride) functional group has excellent coupling capability with the biomaterial. Therefore, when the second substrate 120 is formed by controlling an amount of the polymer having the poly (styrene-maleic anhydride) functional group, adhesion of the biomaterials may be improved.

As shown in FIG. 4, when the first substrate 110 is coupled with the second substrate 120, the biomaterials C formed on the second substrate 120 may be inserted into the micro wells 112 formed in the first substrate 110. The reagents M contained in the micro well 112 may be supplied to the biomaterials C.

In order to maintain the function of the cells, the culture solution is required to be continuously supplied and in order to measure the reaction of the biomaterials C for a specific drug, the specific drug is supplied to the biomaterials C. A toxicity test, chemosensitivity and resitivity test, or the like, for new drug production may be performed by supplying specific drugs.

When various experiments such as cultivating cells or analyzing the characteristics of the biomaterials according to the reagents are performed, the biochip may be disposed for a long period of time in an environment having a range of temperatures, above and below room temperature, or the like. An incubator may need to be supplied with carbon dioxide or be maintained at an appropriate level of humidity during the cell culturing process or the experimental process.

When the reagents such as the culture solution, or the like, are evaporated, the experiment accuracy and efficiency of the biomaterial may be degraded. As the size of the micro well in which the reagents are contained is reduced, the reagents may be evaporated.

However, according to the embodiment of the present invention, water maybe received in the reservoir formed in the first substrate. The water may be evaporated at an appropriate speed at a temperature at which the cell culturing and various experiments are performed, whereby the vapor pressure of the biochip may be controlled. As the vapor pressure of the biochip is controlled, the evaporation of the reagent contained in the micro well may be suppressed and the amount of the reagent may be maintained at an appropriate level.

According to the embodiment of the present invention, the water received in the reservoir may be evaporated through the through holes formed in the barrier ribs.

The biochip according to the embodiment of the present invention may maintain the vapor pressure through the biochip structure itself by forming the reservoir receiving liquid other than the reagent.

When the amount of the reagent is increased by simply increasing only the size of the micro well, the evaporation speed of the reagent may be reduced; however, as the height of the reagent contained in the micro well is reduced, the biomaterial may not be immersed in the reagent accordingly.

However, according to the embodiment of the present invention, the micro well is formed to have an appropriate size so as to immerse the biomaterials therein while maintaining high integration and constantly maintaining the amount of the reagent.

According to the embodiment of the present invention, the micro well and the biomaterial may be arranged in the first substrate and on the second substrate in a highly integrated state. As the biomaterial is in a highly integrated state, several diagnoses may be simultaneously performed and the accuracy of the experimental results may be increased. In addition, the experiment or the diagnosis of the characteristics of the same drug may be simultaneously performed by using various types of biomaterials.

The biochip according to the embodiment of the present invention is configured by coupling the first and second substrates, such that the first and second substrates may be washed separately and the culture solution and the reagent contained in the micro well may be periodically exchanged.

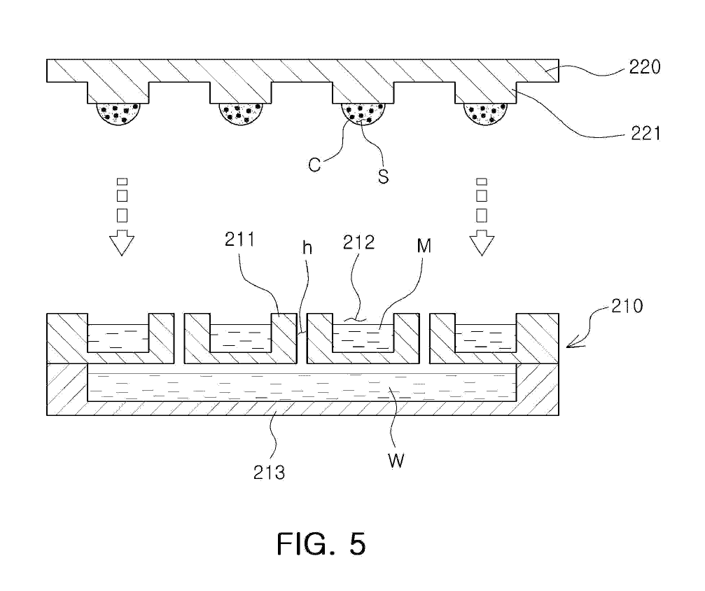

FIG. 5 is a cross-sectional view schematically showing a biochip according to another embodiment of the present invention. Different components other than those described in the above-mentioned embodiments will be mainly described and therefore, a detailed description thereof will be omitted.

Referring to FIG. 5, the biochip according to this embodiment of the present invention may include a first substrate 210 having a plurality of barrier ribs 211, micro wells 212, and a reservoir 213 formed therein and a second substrate 220.

The second substrate 220 may be provided with a plurality of micro fillers 221 having a predetermined interval therebetween. The micro fillers 221 maybe formed on positions corresponding to those of the micro wells 211 formed in the first substrate 210.

The micro filler 221 refers to a structure protruded to a predetermined height from one surface of the second substrate 220 and may be understood as a micro rod or a micro pin. The micro filler 221 may have a three-dimensional structure and a protruding surface of the micro filler 221 may have the biomaterial C attached thereto.

The micro filler 221 may have various heights. For example, the height of the micro filler 221 may be 50 to 500 μm, without being limited thereto. In addition, the shapes of the cross section and the protruding surface of the micro filler are not specially limited.

The reservoir 213 may be formed in the lower portion of the first substrate 210 and may receive liquid. A vapor pressure of the biochip may be maintained by the liquid. Hwere, the liquid may be evaporated at an appropriate speed at a temperature at which various experiments are preformed.

The reservoir 213 may be formed below the micro well 212. The reservoir 213 may be formed to contact the bottom surface of the micro well.

According to the embodiment of the present invention, as shown in FIG. 5, the barrier ribs 211 may be provided with through holes h communicating with the reservoir 213. The liquid received in the reservoir 213 maybe evaporated through the through holes h.

When the first substrate 210 and the second substrate 220 are coupled with each other, the micro fillers 221 formed on the second substrate 220 maybe inserted into the micro wells 211 formed in the first substrate 210.

As described in the embodiment of the present invention, when the biomaterials C are formed on the micro fillers 221, the coupling of the biomaterial C and the reagent M may be improved. In addition, the biomaterials may be easily washed after various experiments are performed, since the biomaterials are attached to the protruding structures, that is, the micro fillers 221.

As described above, according to the embodiment of the present invention, the liquid may be received in the reservoir formed in the first substrate, and the vapor pressure of the biochip may be controlled by the evaporation of the liquid. As the vapor pressure of the biochip is controlled, the evaporation of the reagent contained in the micro well may be suppressed and the amount of the reagent may be maintained at an appropriate level.

As set forth above, a biochip according to embodiments of the present invention can be immersed in various reagents contained in micro wells formed in the first substrate. When the biomaterials are inserted into the micro wells, various reagents can be directly supplied to the biomaterials. Therefore, cells can be cultivated and various experiments can be performed by analyzing the characteristics of the biomaterials.

According to the embodiments of the present invention, the micro wells and the biomaterials can be highly integrated on first and second substrates. As the biomaterials are in a highly integrated state, several diagnoses can be simultaneously performed and the accuracy of the experimental results can be increased. In addition, the experiment or the diagnosis of the characteristics of drugs can be simultaneously performed by forming various types of biomaterials.

According to the embodiments of the present invention, liquid can be received in a reservoir formed in the first substrate, and the vapor pressure of the biochip can be controlled by the evaporation of the liquid. As the vapor pressure of the biochip is controlled, the evaporation of the reagents contained in the micro wells can be suppressed and the amount thereof can be maintained at an appropriate level.

According to the embodiments of the present invention, the water received in the reservoir can be evaporated through through holes formed in the barrier ribs.

The biochip according to the embodiments of the present invention can maintain the vapor pressure by the biochip structure itself by forming the reservoir receiving the liquid other than the reagent.

According to the embodiments of the present invention, the micro wells are formed to have an appropriate size so as to immerse the biomaterials therein while maintaining high integration and constantly maintaining the amount of the reagent.

According to the embodiments of the present invention, micro fillers can be formed on the second substrate, and when the biomaterials are formed on the micro fillers, the coupling of the biomaterials with the reagents can be improved. In addition, the biomaterials can be easily washed after various experiments are performed due to the attachment of the biomaterials to the protruding structures, that is, the micro fillers. Therefore, measurement efficiency and experiment accuracy can be improved.

While the present invention has been shown and described in connection with the embodiments, it will be apparent to those skilled in the art that modifications and variations can be made without departing from the spirit and scope of the invention as defined by the appended claims.

What is claimed is:

1. A biochip, comprising:
a first substrate having micro wells formed to a predetermined depth and partitioned by a plurality of barrier ribs, and a reservoir formed below the micro wells and receiving liquid; and
a second substrate coupled with the first substrate to thereby allow biomaterials provided thereon to be inserted into the micro wells while having a predetermined interval therebetween.

2. The biochip of claim 1, wherein the barrier ribs are provided with through holes communicating with the reservoir.

3. The biochip of claim 1, wherein the reservoir has a single receiving part provided to correspond to the plurality of micro wells.

4. The biochip of claim 1, wherein the reservoir has a plurality of receiving parts corresponding to the plurality of micro wells, respectively.

5. The biochip of claim 1, wherein the reservoir has a plurality of receiving parts, each of which is provided to correspond to the plurality of micro wells.

6. The biochip of claim 1, wherein the liquid is water or deionized water.

7. The biochip of claim 1, wherein the biomaterials are dispersed in a porous dispersion material and are disposed on the second substrate.

8. The biochip of claim 1, wherein the second substrate is provided with a plurality of micro fillers having a predetermined interval therebetween, and
the biomaterials are disposed on one surface of each of the micro fillers.
